(19) United States
(12) Patent Application Publication
(10) Pub. No.: US 2023/0148943 A1
(43) Pub. Date: May 18, 2023

(54) NETWORK ANALYSIS OF ELECTROMYOGRAPHY FOR DIAGNOSTIC AND PROGNOSTIC ASSESSMENT

(71) Applicant: Battelle Memorial Institute, Columbus, OH (US)

(72) Inventors: Eric C. Meyers, Columbus, OH (US); Nicholas J. Tacca, Columbus, OH (US); David Gabrieli, Grandview Heights, OH (US); Michael Darrow, Missouri City, TX (US); Lauren R. Wengerd, Columbus, OH (US); David A. Friedenberg, Worthington, OH (US); Bryan R. Schlink, Westerville, OH (US)

(21) Appl. No.: 18/054,232

(22) Filed: Nov. 10, 2022

Related U.S. Application Data
(60) Provisional application No. 63/279,101, filed on Nov. 13, 2021.

(51) Int. Cl. A61B 5/00; A61B 5/296; A61B 5/256; A61B 5/389; A61B 5/11; A61N 1/36

(52) U.S. Cl. CPC A61B 5/407; A61B 5/296; A61B 5/256; A61B 5/389; A61B 5/6804; A61B 5/7246; A61B 5/4848; A61B 5/1124; A61N 1/36003; A61B 2562/046

(57) ABSTRACT

In a method of neurological assessment, multichannel electromyography (EMG) data are acquired for an anatomical region. A pairwise EMG channel-EMG channel similarity matrix is generated from the acquired multichannel EMG data. Network analysis is performed on the similarity matrix to generate a network representing the similarity matrix. One or more metrics of the network are computed. One or more biomarkers are determined for the anatomical region based on the one or more metrics. In another method, EMG data are acquired using an electrode array contacting skin of a target anatomy, the EMG data are processed to produce reduced-dimensionality data; and time-invariant muscle synergies and corresponding time-varying activation functions are determined in the reduced-dimensionality data.

### CROSS-REFERENCE TO RELATED APPLICATIONS

This application claims the benefit of U.S. Provisional Application No. 63/279,101 filed Nov. 13, 2021 and titled “NETWORK ANALYSIS OF ELECTROMYOGRAPHY FOR DIAGNOSTIC AND PROGNOSTIC ASSESSMENT”, which is incorporated herein by reference in its entirety.

### BACKGROUND

The following relates to the neurological assessment, neurological treatment monitoring, stroke assessment and therapy, spinal cord injury (SCI) assessment and therapy, and to the like.

Electromyography (EMG) measured via transcutaneous (i.e. surface) electrodes has been used to diagnose muscular and nerve disorders such as amyotrophic lateral sclerosis (ALS) and Guillain-Barre syndrome. Transcutaneous EMG provides information about EMG activity proximate to the skin, but its use has been limited in diseases where nerve damage is localized within the central nervous system, such as spinal cord injury or stroke.

### BRIEF SUMMARY

In accordance with some illustrative embodiments disclosed herein, a method of neurological assessment includes: acquiring multichannel electromyography (EMG) data for an anatomical region; generating a pairwise EMG channel-EMG channel similarity matrix from the acquired multichannel EMG data; performing network analysis on the similarity matrix to generate a network representing the similarity matrix; computing one or more metrics of the network; and determining one or more biomarkers for the anatomical region based on the one or more metrics.

In accordance with some illustrative embodiments disclosed herein, a method comprises: acquiring EMG data using an electrode array contacting skin of a target anatomy; processing the EMG data to produce reduced-dimensionality data; and determining time-invariant muscle synergies and corresponding time-varying activation functions in the reduced-dimensionality data. By way of non-limiting illustrative example, the processing of the EMG data to produce reduced-dimensionality data comprises processing the EMG data using one or more of: non-negative matrix factorization (NMF); factor analysis; principal component analysis (PCA), independent component analysis (ICA), an autoencoder, a generative adversarial network, or a combination thereof. In some embodiments, the determining of the time-invariant muscle synergies includes determining a number of muscle synergies based on reconstruction of the acquired EMG data from the reduced-dimensionality data via the muscle synergies and muscle synergy activation.

In some embodiments, the method of the immediately preceding paragraph further includes: repeating the acquiring, processing, and determining for different anatomical targets and/or different subjects; and comparing the determined muscle synergies of the different anatomical targets and/or different groups of people to identify target muscles and/or functional movements for rehabilitation training. In some embodiments, the method of the immediately preceding paragraph further includes: repeating the acquiring, processing, and determining for multiple sessions; and correlating the determined muscle synergies over the multiple sessions with changes to corticospinal reorganization to assess motor recovery. In some embodiments, the method of the immediately preceding paragraph further includes: determining a starting stimulation pattern based on the determined muscle synergies; and performing functional electrical stimulation (FES) or neuromuscular electrical stimulation (NMES) on the target anatomy using the starting stimulation pattern.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically shows a system for neurological assessment using network analysis of surface electromyography (EMG) signals.

FIGS. 2-6 diagrammatically show illustrative surface EMG data collection for generating similarity matrices for stroke patients.

FIGS. 7-9 present some results obtained for network analyses performed on binarized similarity matrices.

FIGS. 10-14 present some results obtained for network analyses performed on weighted similarity matrices,

FIGS. 15A and 15B present results using a coherence network analysis.

FIGS. 16-21 and 22A-22B present some further experimental results.

### DETAILED DESCRIPTION

As recognized herein, EMG provides an advantageous modality for understanding the muscular system, which is key to assessing and restoring functional independence. While functional clinical assessments of improved motility can provide quantitative metrics to determine progress, they can lack the granularity to properly differentiate subjects and can have variable results based on operator.

As disclosed herein, additional quantitative information can be derived using an EMG sleeve as a signal modality for assessment. Network theory is used to analyze the synergies between different areas of the forearm (or, more generally, anatomy to be assessed), and is used to quantify differences in functional capabilities in disease and during rehabilitation. Muscle synergies are characteristic patterns of activations across multiple muscles groups that individually scale and combine to enable complex movements. Complex network theory facilitates understanding of the relationships between different recorded EMG signals, and is used to produce a set of quantifiable metrics that can identify differences in subjects with prior stroke and differentiate levels of impairment.

In an illustrative embodiment, EMG recordings are taken from functional movement studies and used to build relationships between each pair of electrode signals. This relationship can be from correlation, phase synchronization, or coherence along the time series, by way of some nonlimiting illustrative examples. A network graph is created that uses these relationships between electrode signals as connections and the electrodes as nodes. Complex network metrics are used to analyze the graph and relate metrics to task performance or clinical measures. Some such embodiments have been reduced to practice using data from a stroke study, demonstrating that the ability to decode a subject's movement intent may be related to network efficiency, with easier-to-decode subjects having more local microstructures similar to their able body counterparts.

In various embodiments, complex network analysis of EMG is used to provide prognostic capabilities for subjects with an upper limb impairment. An EMG sleeve or other garment is worn on the arm or other anatomy to be assessed. The EMG sleeve includes an array of electrodes connected to channels of an EMG amplifier to measure EMG signals, Analyses of the EMG signals using network theory allows for assessment of underlying muscular activity, which can be beneficial in various clinical situations such as assessing neurological status of subjects that have limited mobility after stroke or spinal cord injury. In some illustrative embodiments, relationships between the signals in different areas of the forearm are determined to build graphs or networks, and complex network analysis is used to identify indicators of impairment level and functional recovery.

The disclosed approaches advantageously leverage network analysis performed on a similarity matrix capturing pairwise surface EMG channel-EMG channel comparisons to derive relationships between different recorded EMG signals that, as recognized herein, is indicative of nerve damage that is localized within the central nervous system, such as is commonly the case in spinal cord injury (SCI) or stroke patients. In general, higher network connectivity as indicated by network and/or nodal metrics is expected to correlate with improved neurological coordination and hence less neurological impairment at the level of the assessed portion of the central nervous system. More particularly, local (i.e. nodal) and global (i.e. network) metrics of the network analysis are recognized herein to be indicative of the way the muscle networks are arranged, such as whether the muscle networks have dense interconnected clusters of related electrodes, or a central electrode that was the main connection between different sections of the network. By investigating whether networks split into one central, core section with some electrodes that were more on the periphery, or if networks were functionally organized to be efficient, the level of impairment can be assessed.

The disclosed approaches thus enable assessment of nerve damage localized within the central nervous system using transcutaneous (i.e. surface) EMG signals. In illustrative embodiments disclosed herein, an EMG sleeve or other garment with a high density surface electrode array provides a rapid and convenient way to provide sufficient data from a range of locations around the portion of the central nervous system being assessed to perform the network analysis.

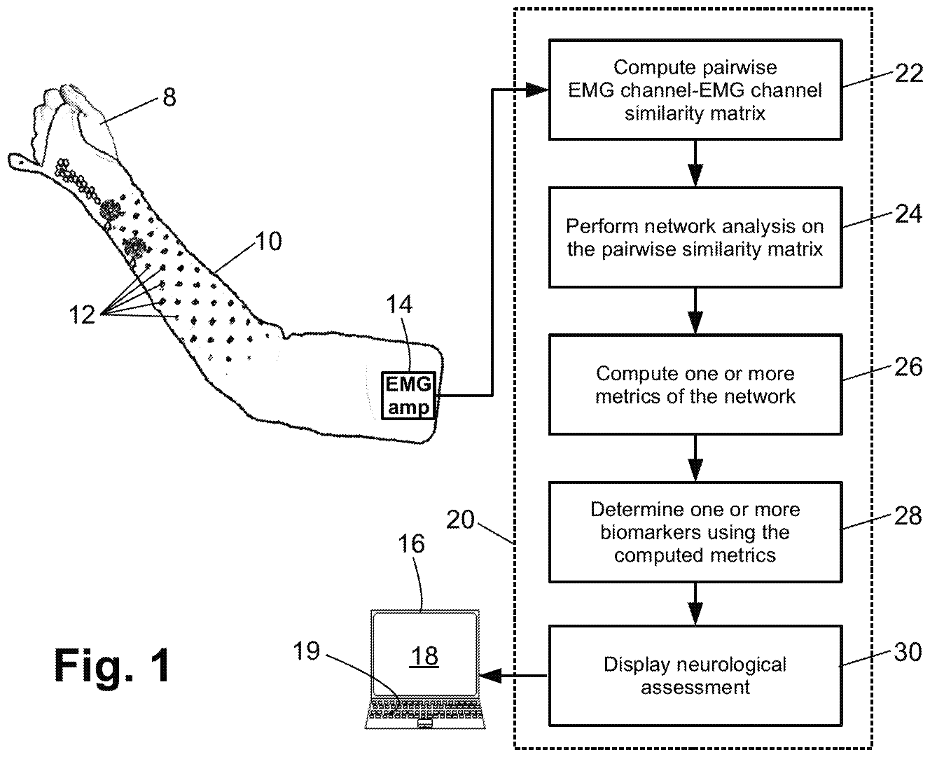

With reference to FIG. 1, in an illustrative embodiment an electromyography (EMG) measurement garment 10, such as an illustrative EMG sleeve 10, is worn on the forearm (or other target anatomy to be assessed) of a subject 8 (e.g., a patient, rehabilitation subject, or so forth; indicated in FIG. 1 by only a portion of the hand of the subject 8 not covered by the EMG sleeve 10). The EMG measurement sleeve 10 includes transcutaneous (i.e. surface) electrodes 12 distributed over the inside surface of the arm (or, more generally, over the surface of a target anatomy such as an arm, leg, torso, or other anatomy arranged to contact the skin of that target anatomy). It should be noted that the electrodes 12 are diagrammatically shown in FIG. 1—in practice, the electrodes 12 are arranged on an inner surface of the sleeve 10 so as to contact the skin of the target anatomy when the garment 10 is donned on the target anatomy. (The inner surface of the garment 10 is the surface of the garment 10 that faces toward the skin of the anatomy, e.g. illustrative arm, when the garment 10 is worn on the anatomy). More generally, the electrodes 12 are arranged on an inner surface of a garment 10 to contact skin of the target anatomy when the garment 10 is worn on the target anatomy. Use of the illustrative sleeve or other garment 10 that is worn on the target anatomy is a convenient way to quickly position a large number of electrodes 12 distributed over the skin of the target anatomy, so as to provide sufficient surface EMG data for the network analysis from numerous points around the portion of the central nervous system to be analyzed. However, it is contemplated to apply the electrodes to the skin using another approach, such as applying individual transcutaneous electrodes to the skin using electrically conductive adhesive to adhere the individual electrodes to the skin.

A multichannel EMG amplifier 14 is operatively connected to the electrodes 12 to read the EMG signals. The garment 10 preferably includes a number of electrodes that is sufficient to provide adequate data for the subsequent network analysis. For example, in some nonlimiting illustrative embodiments, the garment 10 includes at least 100 electrodes, and more preferably 150 or more electrodes, which are distributed over the surface of the arm or other target anatomy when the garment is worn on the target anatomy. This enables multichannel high-density EMG (HD-EMG) measurements for constructing a detailed pairwise similarity matrix comparing EMG channel pairs.

The multichannel EMG amplifier 14 may have a separate channel for each electrode 12 so that the number of channels of the EMG amplifier 14 equals the number of electrodes 12, Alternatively, to reduce hardware costs, the EMG amplifier 14 may use time-dimension multiplexing (TDM) to enable each channel of the EMG amplifier 14 to read multiple electrodes 12. In variant embodiments, it is contemplated to for a single EMG channel to include multiple electrodes in a contiguous region to increase (spatially averaged) EMG signal strength, albeit at the cost of reduced spatial resolution. Because the EMG signals are of low intensity, in some embodiments the EMG amplifier 14 (or at least a front-end amplifier circuit portion thereof) may be integrated with the sleeve or other garment 10 as diagrammatically shown in FIG. 1 to reduce the signal path lengths from the electrodes to the EMG amplifier 14.

With continuing reference to FIG. 1, an electronic processing device 16, optionally including a display 18, performs a neurological assessment method 20. In FIG. 1 the electronic processing device 16 is shown as an illustrative implementation as a computer 16 having the display 18 and an optional keyboard and/or other user input device 19. More generally, the electronic processing device 16 may comprise a notebook computer, desktop computer, a mobile device such as a cellular telephone (cellphone) or tablet computer, or so forth, that is operatively connected to read EMG signals from the EMG amplifier 14 and that includes an electronic processor (not shown; e.g. a microprocessor or microcontroller) that is programmed by instructions stored on a non-transitory storage medium (not shown) and readable and executable by the electronic processing device 16 to perform the neurological assessment method 20. The non-transitory storage medium may, for example, comprise a hard drive or other magnetic storage medium, a flash memory, solid state drive (SSD) or other electronic memory, an optical disk or other optical memory, various combinations thereof, and/or so forth.

In the neurological assessment method 20, an operation 22 computes a pairwise EMG channel-EMG channel similarity matrix. The similarity matrix is an N×N matrix where N is the number of EMG channels. Each element (Chᵢ,Chⱼ) of the similarity matrix stores the value of a similarity metric S(Chᵢ,Chⱼ) measuring similarity between EMG channel Chᵢ and EMG channel Chⱼ (where 1≤Chᵢ≤N and 1≤Chⱼ≤N) The diagonal of the similarity matrix stores elements S(Chᵢ,Chⱼ)=1 if the similarity metric S outputs 1 for identical elements (this is an example; a similarity metric S that outputs some value other than 1 for identical elements is also contemplated). In some embodiments, the similarity matrix is a symmetric matrix, which is obtained if the similarity metrics S(Chᵢ,Chⱼ)=S(Chᵢ,Chⱼ) for all (Chᵢ,Chⱼ) pairs. In one nonlimiting illustrative embodiment, the similarity metric S(Chᵢ,Chⱼ) is a correlation coefficient for the pair of EMG channels Chᵢ and Chⱼ, or a coherence coefficient for the pair of EMG channels Chᵢ and Chⱼ.

In an operation 24, a network analysis is performed on the pairwise similarity matrix. The network analysis can, by way of some nonlimiting illustrative examples, comprise non-negative matrix factorization (NMF) of the similarity matrix, computing a connectivity matrix, performing a coherence network analysis method, performing a correlation network analysis method, various combinations thereof, or so forth. The output of the operation 24 is a network representation of the similarity matrix. In an operation 26, one or more metrics of the network are computed. These may include network metrics which are characteristic of the whole network, and/or nodal metrics which are characteristic of individual nodes of the network.

By way of some nonlimiting illustrative examples, some suitable network metrics may include density metrics measuring the fraction of present connections to possible connections, global efficiency metrics measuring the average inverse shortest path length in the network, characteristic path length metrics measuring the average shortest path length in the network, and/or core periphery q-stat metrics. Regarding the latter network metric, the core/periphery subdivision is a partition of the network into two non-overlapping groups of nodes: a core group and a periphery group, in a way that maximizes the number (or weight) of within core-group edges, and minimizes the number/weight of within periphery-group edges.

By way of some nonlimiting illustrative examples, some suitable nodal metrics for a node of the network may include degree metrics measuring the number of links connected to the node, clustering coefficient metrics measuring the fraction of node's neighbors that are neighbors of each other, local efficiency metrics measuring the global efficiency computed on the neighborhood of the node, and/or betweenness centrality metrics measuring the fraction of all shortest paths in the network that contain a given node.

In an operation 28, one or more neurological biomarkers are determined using the computed metrics of the network. The set of metrics output by the operation 26 are used, in one nonlimiting illustrative example, to identify differences in subjects with prior stroke and differentiate levels of impairment. In one embodiment, the operation 28 is performed by an artificial neural network (ANN) or other machine learning (ML) component that is trained on a corpus of labeled training examples each comprising values for the set of metrics generated by operations 22, 24, and 26 for a historical patient labeled by a “ground truth” value of the neurological biomarker determined for that historical patient by a qualified neurologist or the like. Rather than a trained ML model, less computationally complex approaches can be used, such as analyzing the training corpus to determine a threshold on a metric of the network such as connections of the similarity matrix for distinguishing between patients with versus without a certain biomarker. Again, these are merely illustrative examples.

With continuing reference to FIG. 1, in an operation 30, the neurological assessment of the patient is displayed, for example by displaying natural language text representations of clinical significance of the biomarkers determined in the operation 28 on the display 18 of the electronic processing device 16.

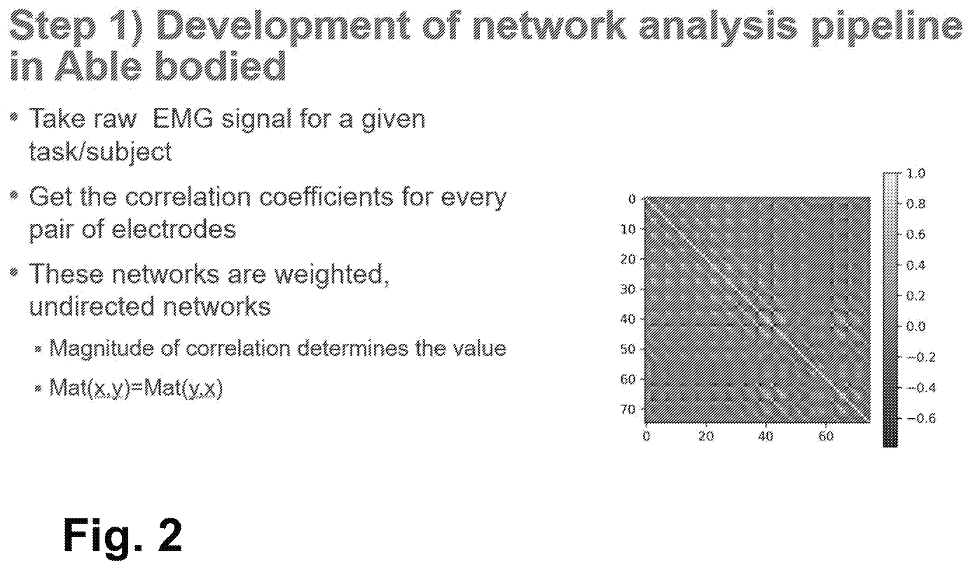

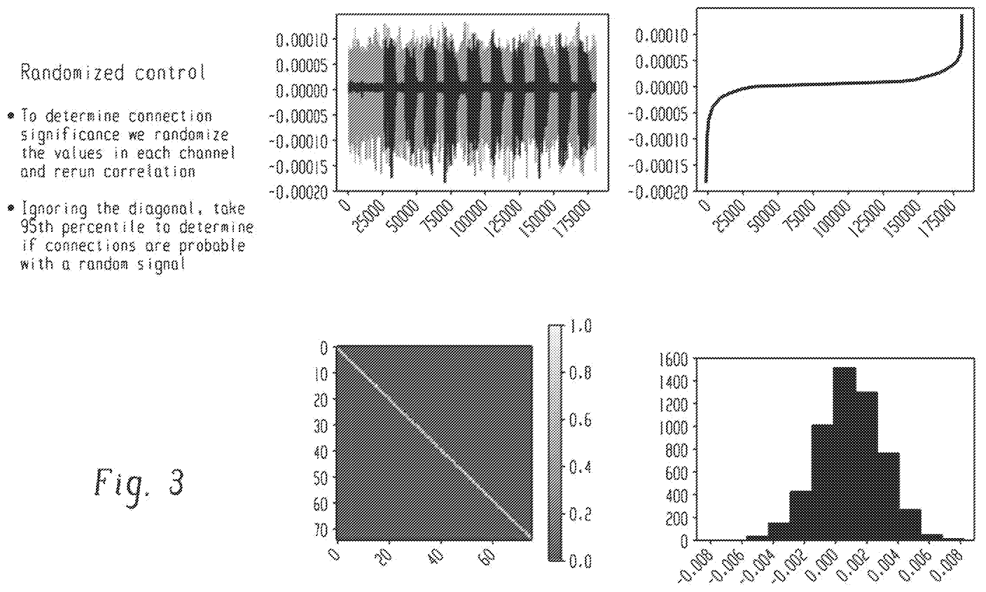

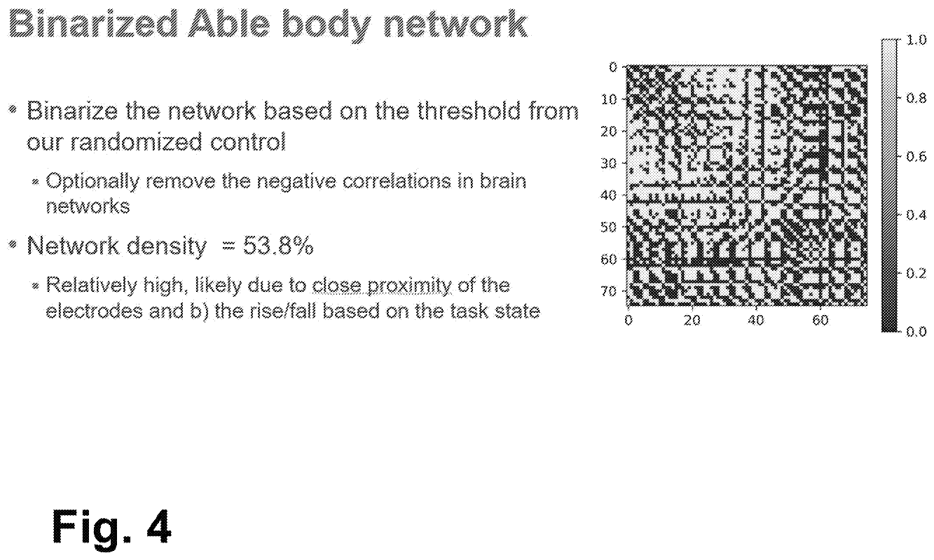

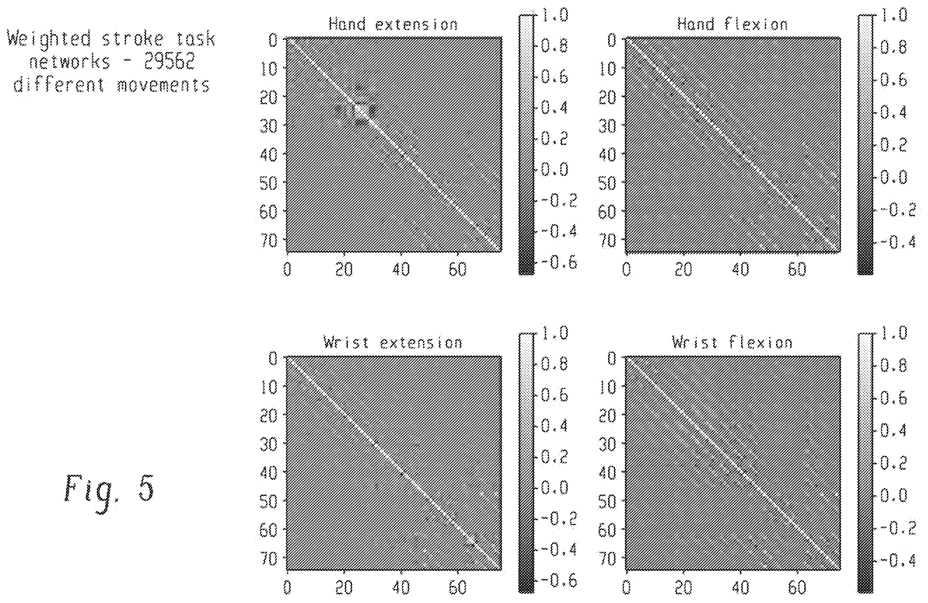

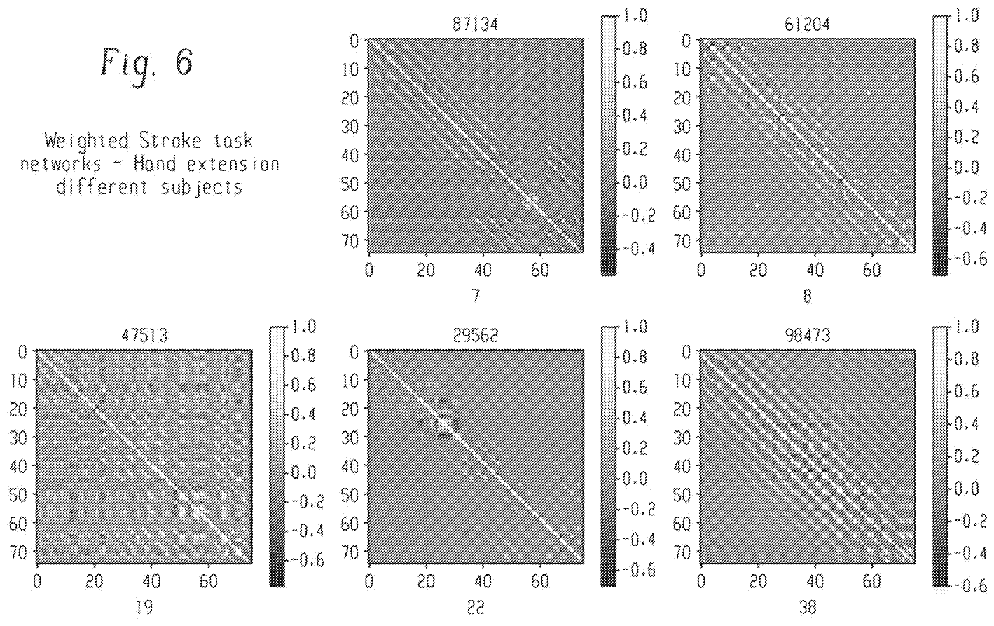

With reference now to FIGS. 2-8 (Example 1), some illustrative surface EMG data collection for generating similarity matrices for stroke patients is shown. The data are for assessment of EMG signals measured using an EMG sleeve similar to the EMG sleeve 10 of FIG. 1 during motions including: hand extension, hand flexion, wrist extension, and wrist flexion. FIG. 2 illustrates the similarity matrix computation. FIG. 3 illustrates use of a randomized control for assessing connection significance as a biomarker. FIG. 4 illustrates binarization of a similarity matrix which is used in some nonlimiting illustrative embodiments. FIGS. 5 and 6 illustrate some examples of similarity matrices obtained for stroke patients,

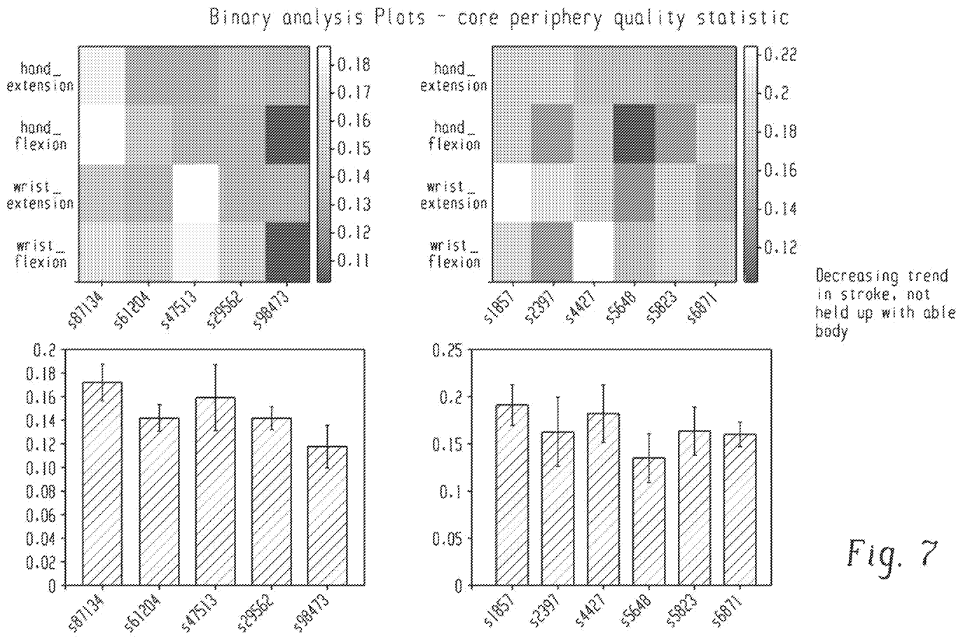

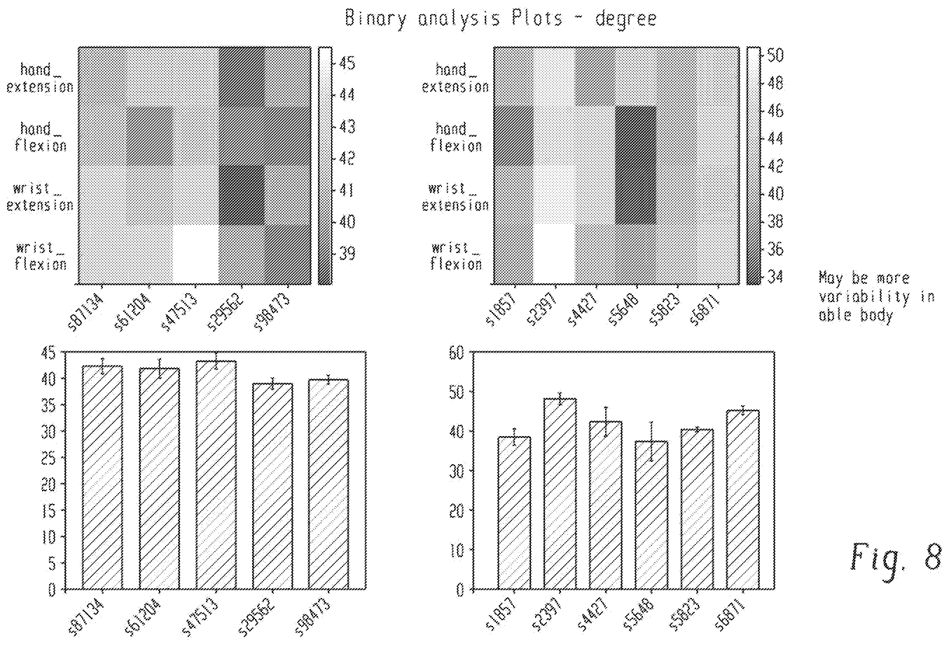

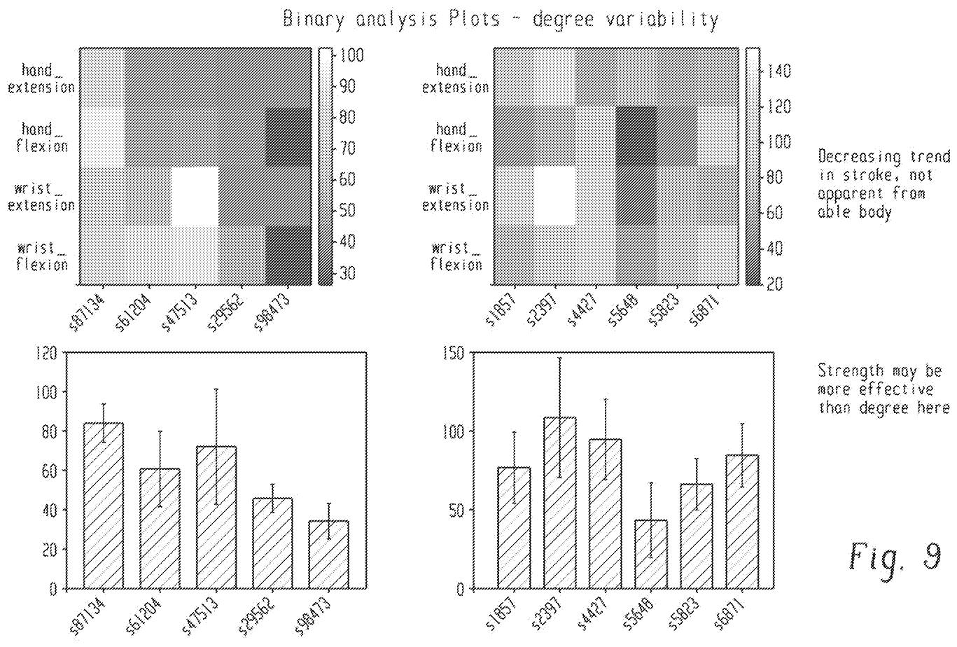

FIGS. 7-9 present some results obtained for network analyses performed on binarized similarity matrices (see FIG. 4). FIG. 7 presents results using the core periphery quality statistic network metric. FIG. 8 presents results using the degree nodal metric. FIG. 9 presents results for degree variability. The binary analysis was used in these experiments mainly as a control to ensure operability and to obtain some preliminary results. Based on these preliminary results, the core periphery structure q-stat appears to have higher values with more severe disability, indicating the core muscular function is less flexible overall. Motor units (MU) are either in or out regardless of movement, where able body have more variable control. Potentially flexors are highly connected to all other areas as they coactivate regardless of movement.

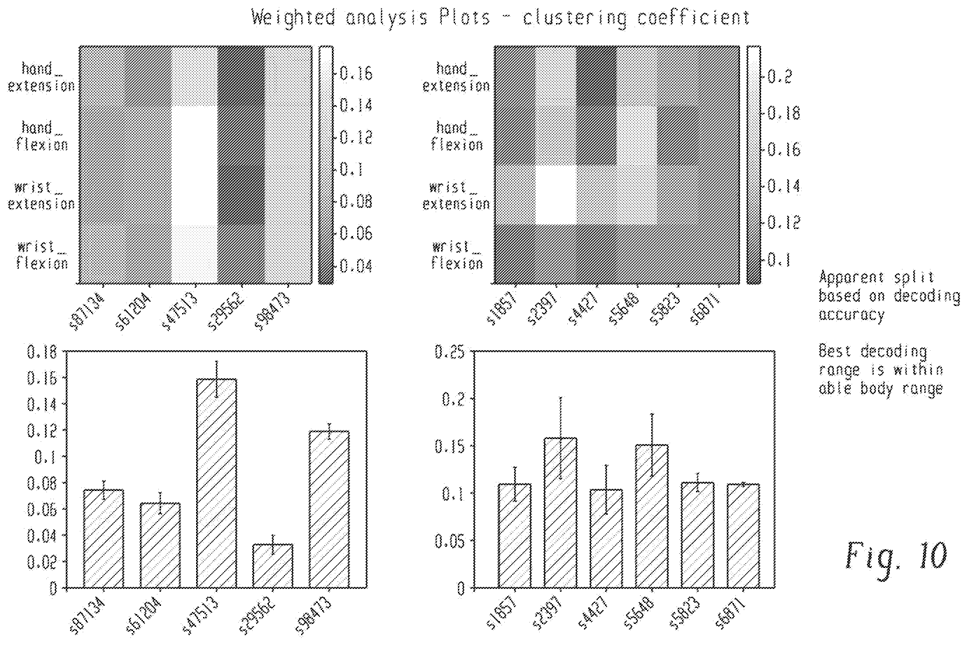

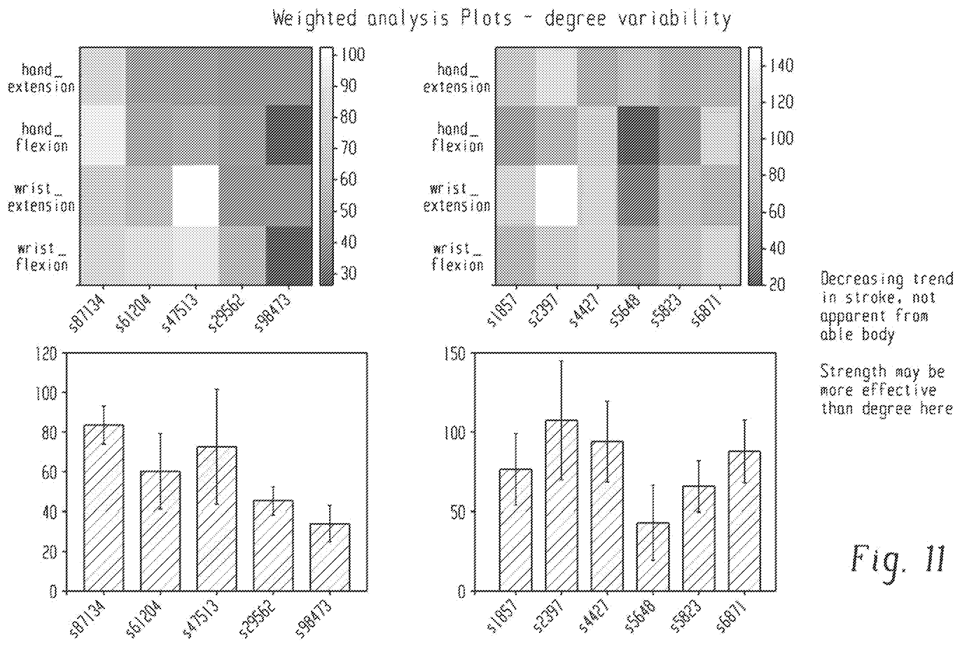

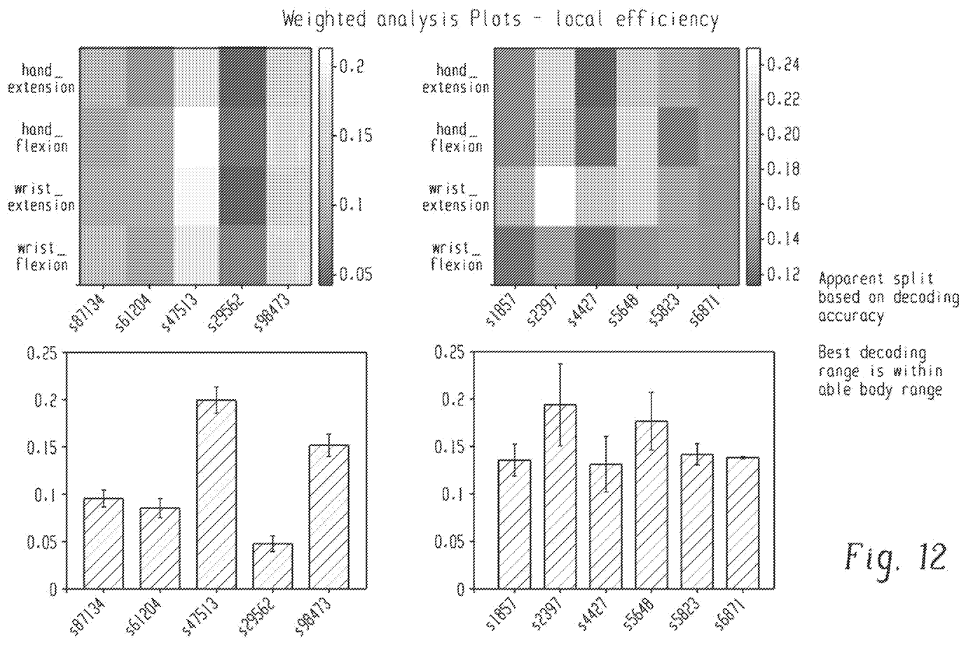

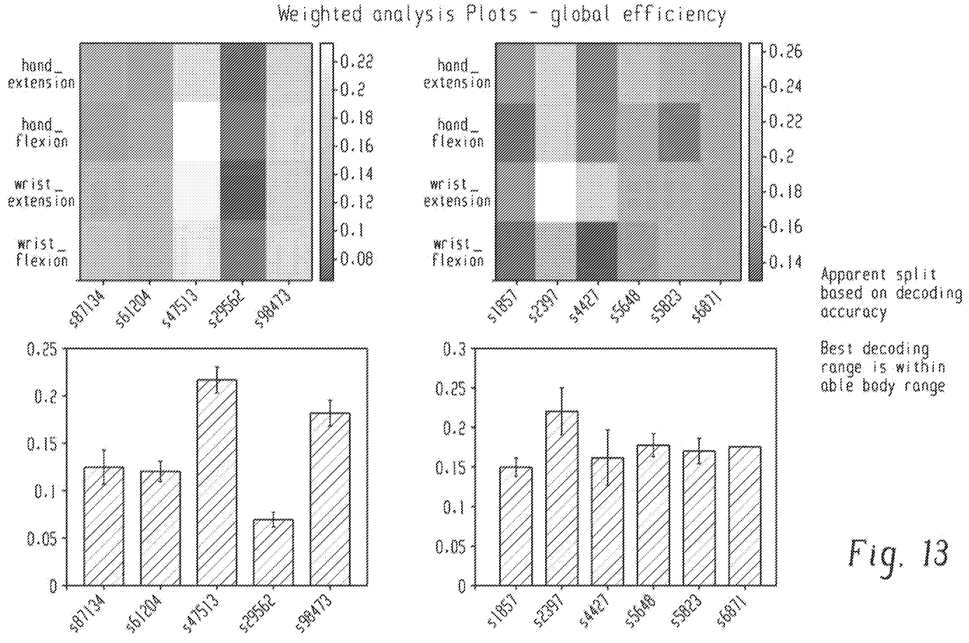

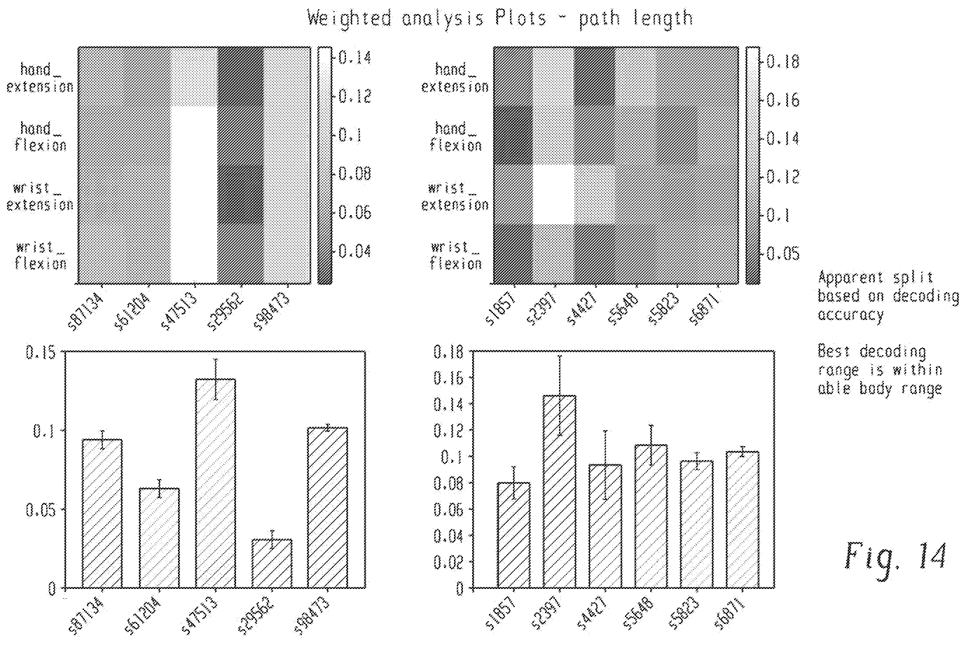

FIGS. 10-14 present some results obtained for network analyses performed on weighted similarity matrices (see FIGS. 5 and 6), FIG. 10 presents results using the clustering coefficient nodal metric. FIG. 11 presents results using degree variability. FIG. 12 presents results using the local efficiency nodal metric. FIG. 13 presents results using the global efficiency network metric. FIG. 14 presents results using the path length network metric. The 29k data appears to be anomalous in the global/local efficiency, clustering coefficient, and path length analyses. Efficiency, clustering coefficient, and path length are related metrics. As there is a physically embedded and consistent scale network, these metrics can be thought of as a global vs local indicators. Higher efficiency, clustering coefficient, path length should indicate the local properties are stronger in these networks. Higher impairment would likely entail global activation with less nuanced local microstructure to create a movement.

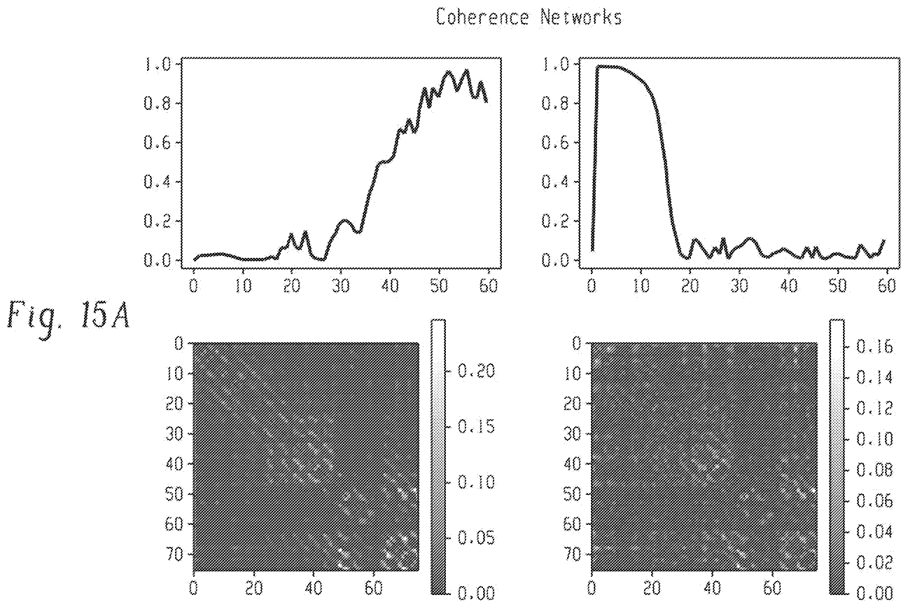

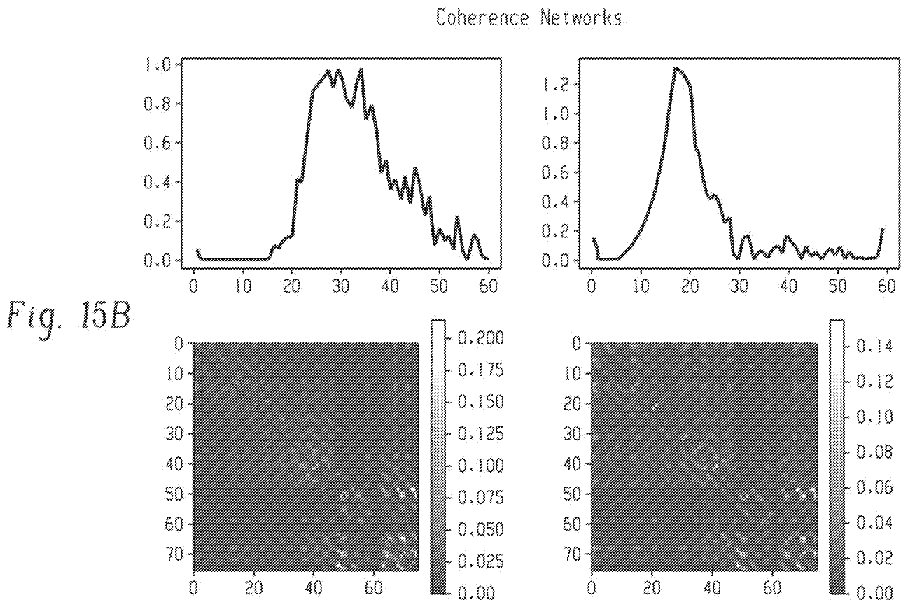

With reference to FIG. 15A and FIG. 15B, results are presented using a coherence network analysis. The presented data are for 29k and have not been updated with averaged NMF models over all subjects. The processing includes high-pass filtering (>20 Hz) followed by band limiting the coherence to 0-60 Hz for creation of the NMF filter.

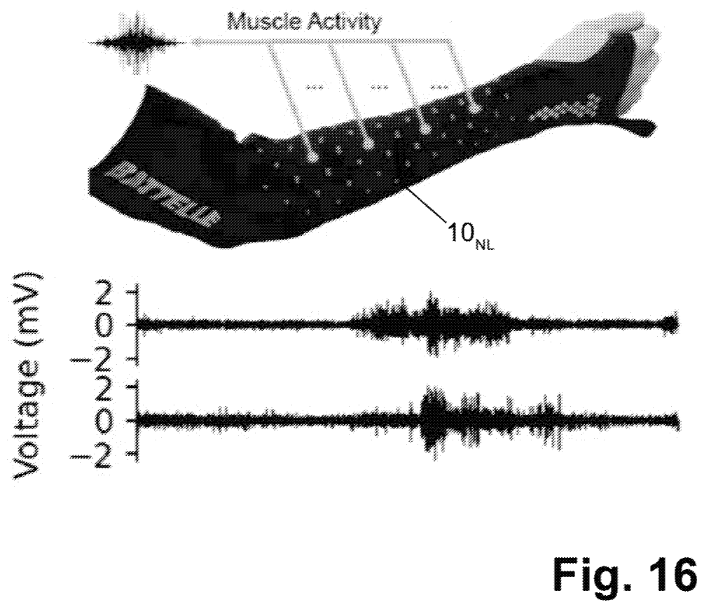

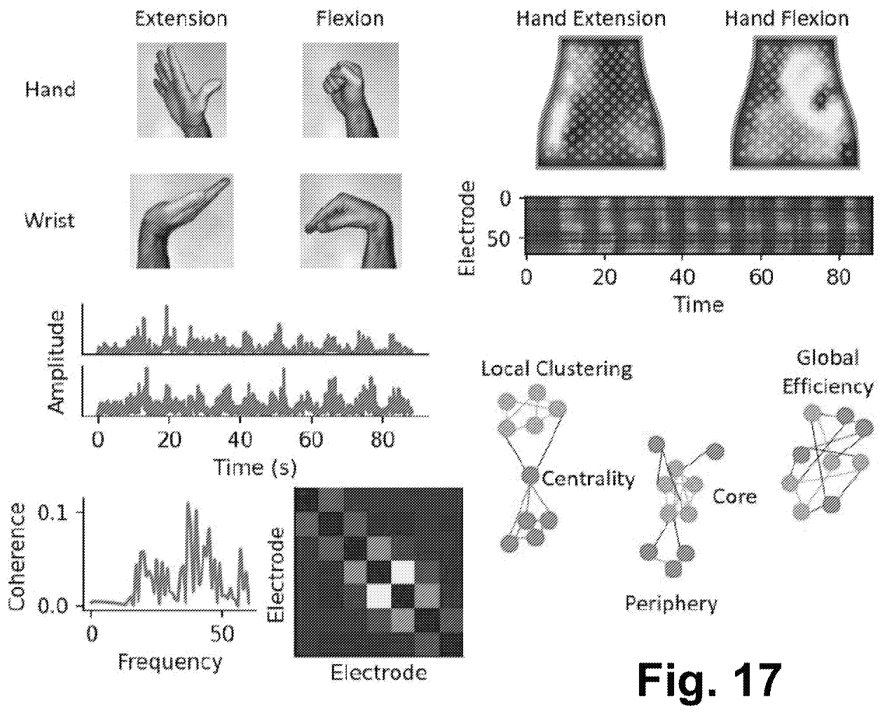

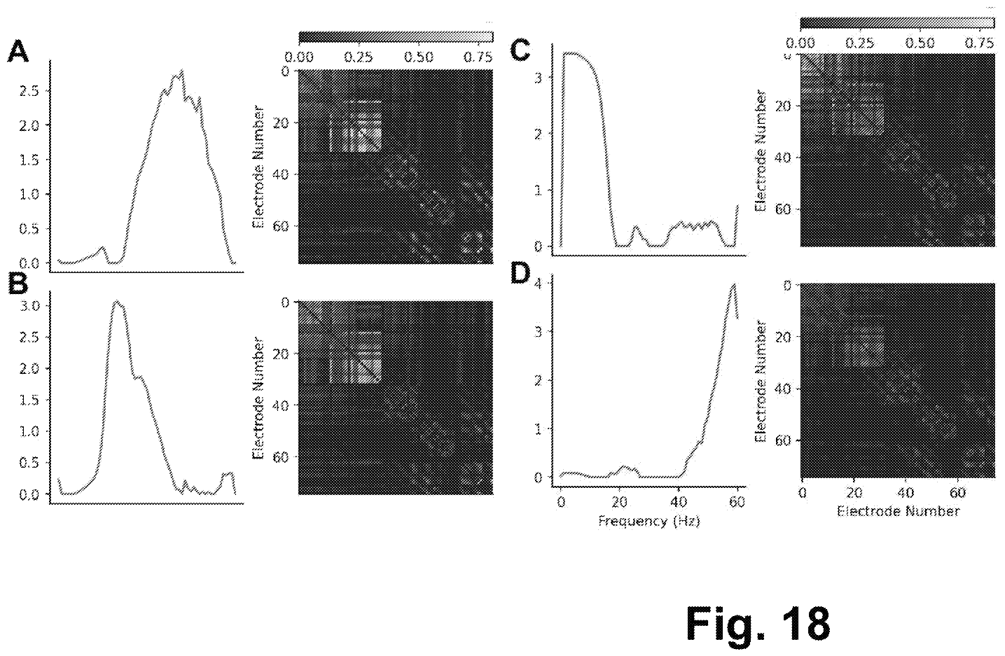

With reference to FIGS. 16-20, some further experimental results are shown. FIG. 16 illustrates the experimental setup using the NeuroLife Sleeve 10_{NL} (a suitable embodiment of the EMG sleeve 10 of FIG. 1, which includes 150-electrode surface EMG garment that covers the forearm). FIG. 16 also plots as a function of time of two illustrative EMG channel signals (measured in voltage, mV, in this example). The example of FIGS. 16-20 relates to assessing chronic stroke survivors that have impaired hand function adversely impacting their independence and ability to perform activities of daily living (ADL). For the study of FIGS. 16-20, six chronic stroke survivors were recruited to perform four movements, namely hand extension, hand flexion, wrist extension and wrist flexion (see FIG. 17, upper left), and have their muscular activity recorded by the NeuroLife Sleeve 10_{NL}. FIG. 17, upper right shows an example of EMG signals over the area of the NeuroLife Sleeve 10_{NL} for a Hand Extension movement and for a Hand Flexion movement, as two illustrative examples, Recorded signals were assessed using non-negative matrix factorization (NMF) of inter-electrode coherence (see Boonstra et al., “Muscle networks: Connectivity analysis of EMG activity during postural control”, Sci Rep, 5, 17830 (2016)), followed by construction of network graphs for each subject and movement (see FIG. 17, lower left). Networks were compared using network graph metrics to assess differences in local and global topology (see Rubinov et al., “Complex network measures of brain connectivity: Uses and interpretations”, NeuroImage, 52, 1059-69 (2010)). Results are presented in FIGS. 18-20 as next described.

FIG. 18 illustrates networks showing muscular coherence varies by frequency content. In this analysis, the EMG signal was decomposed into four distinct components using NMF. The spectra consisted of: a broad peak centered at 42 Hz (FIG. 18(A)); a narrow peak centered at 20 Hz (FIG. 18(B)); a low frequency component (FIG. 18(C)); and a high frequency component (FIG. 18(D)). The corresponding connectivity matrices show distinct topology in each of the extracted spectra. Mean coherence strength was significantly different across frequency spectra (p=0.021). These data indicate that coherence networks appear to be sensitive to neurological deficits caused by a stroke.

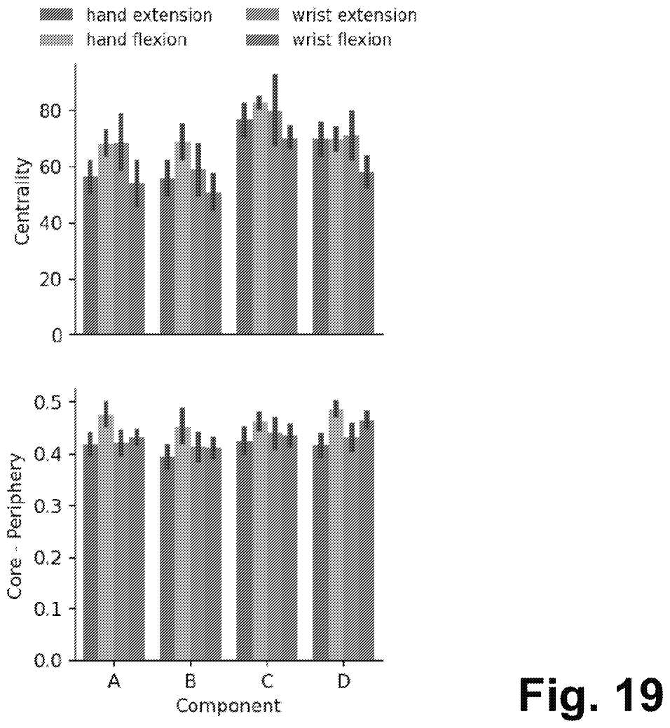

FIG. 19 illustrates that functional organization of musculature can be characterized through network metrics. Centrality and core-periphery structure show significant main effect for movement intent (p=0.027 and p=0.006, respectively). Centrality quantifies the presence of network hubs, or regions that have characteristically high muscular synergy with other regions. Higher core-periphery structure in flexor movements appear to be indicative of sensitivity to spasticity. Notably, centrality measures the hub connection between different regions, which are believed to be areas of muscle that are activating inadvertently in multiple movements. Similarly, flexor movements are found to have these large core networks, which could be from overactive spastic muscle activity during those movements.

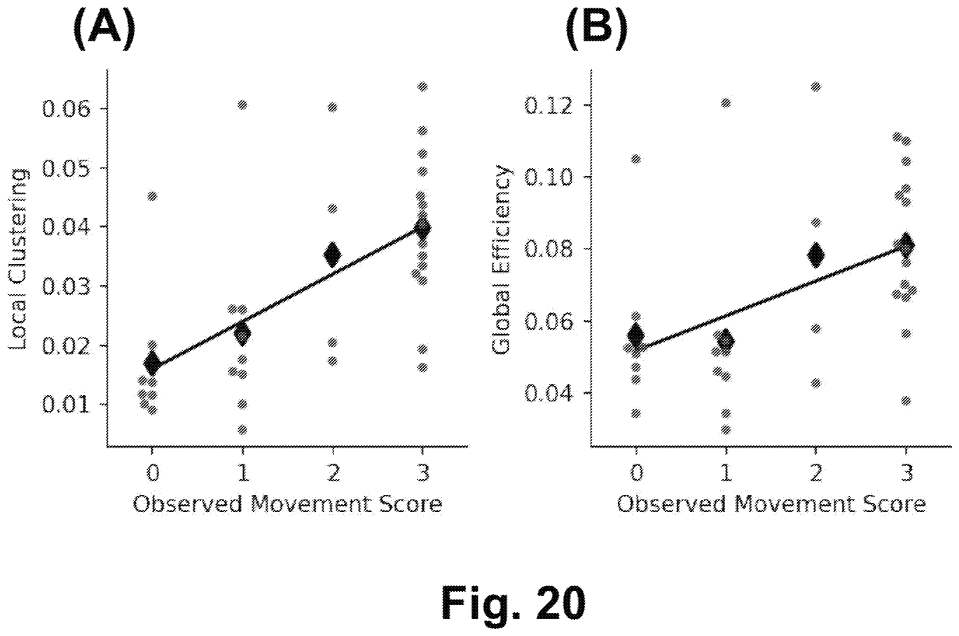

FIG. 20 illustrates that participant observed movement score relates to deficits in local clustering and global efficiency. Observed movement scores were tracked for each participant and movement (0—no movement, 3— normal movement). Significant relationships are seen between observed movement score and both local clustering (FIG. 20(A), p<0.001) and global efficiency (FIG. 20(B), p=0.006) in the low frequency spectra. These results indicate that stroke participant muscular networks are deficient in both local microstructure and global muscular coordination. Notably, in the low frequency band the network metrics related to movement scores in stroke and able body participants, indicating that network metrics are sensitive to impairments and are a suitable quantitative measure of performance.

As disclosed herein, the wearable sleeve 10 with the embedded high-density electrode array 12 provides for concurrent EMG recording and FES delivery. Using the high-density EMG array 12, the underlying muscle synergies of the forearm are extracted. These are the fundamental building blocks of motor control, and those extracted synergies are used to create unique FES patterns for each individual. It is expected that by providing physiologically-relevant feedback to the central nervous system (CNS) through muscle synergy-based FES, this approach more effectively engages the sensorimotor system to further enhance recovery from a stroke or other neurological damage, and is expected to improve outcomes and quality of life for persons living with stroke impairment.

Muscle synergies are characteristic patterns of activations across multiple muscles groups that individually scale and combine to enable complex movements. Following a stroke, muscle synergies in the affected arm are altered while the muscle synergies of the unaffected arm remain unchanged. In some embodiments, the muscle synergies from the unimpaired arm are used to shape rehabilitation of the impaired arm, which is expected to provide a promising method for personalizing stroke therapy. Impaired function of hand and wrist significantly contribute to reductions in quality of life experienced by stroke survivors and are currently a top unmet need. In embodiments disclosed herein, the high-density forearm electrode array 12 of the sleeve 10 (for example, with 150 embedded electrodes in one illustrative example) can both record muscle activity through EMG and deliver FES through the same electrodes. This platform is leveraged herein to extract complex forearm muscle synergies from the unimpaired arm and encode personalized FES patterns delivered to the impaired arm for each subject. This method enhances the physiological relevance of the FES-induced feedback with the goal of improving long-term outcomes.

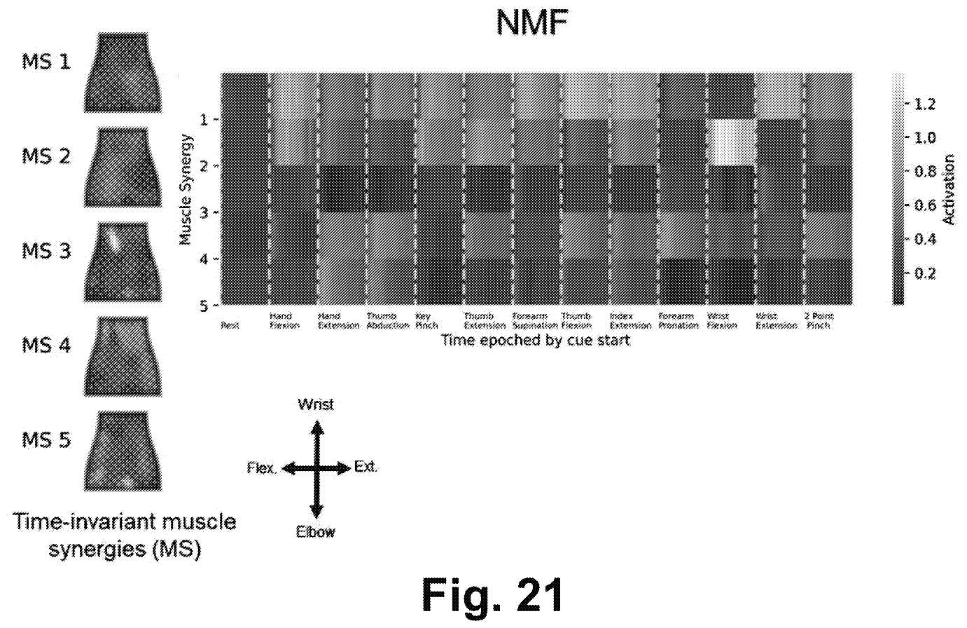

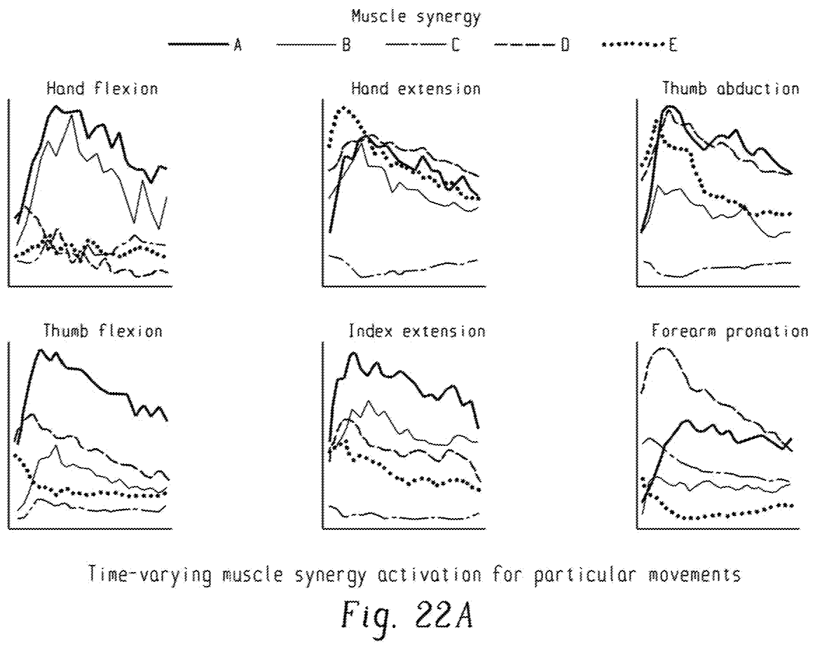

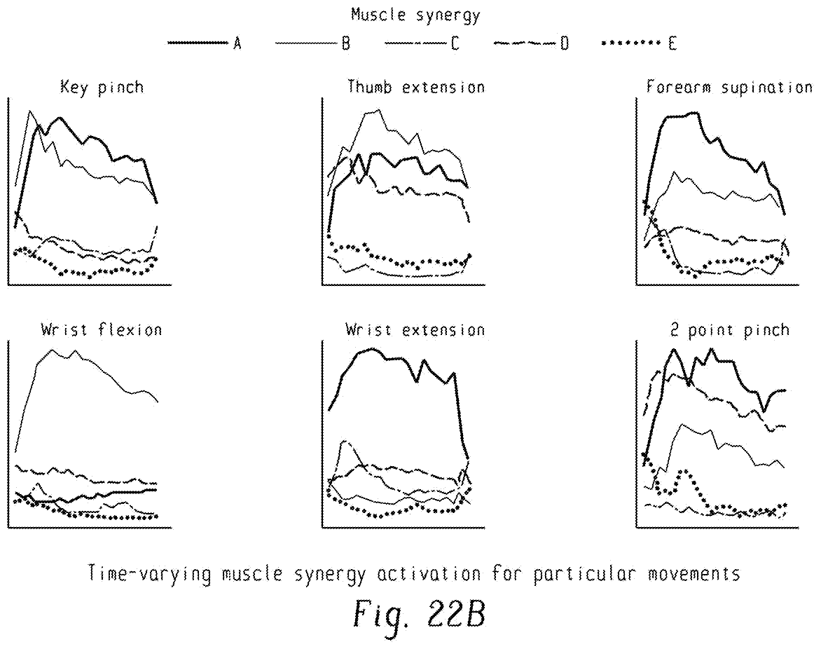

With reference to FIGS. 21 and 22A-22B, a visual representation is shown of an actually performed experiment in which muscle synergies were extracted with non-negative matrix factorization (NMF, FIG. 21) and the time-varying activation of the muscle synergies (FIGS. 22A-22B) for a stroke participant across 12 functional movements (hand flexion, hand extension, thumb abduction, key pinch, thumb extension, forearm supination, thumb flexion, index extension, forearm pronation, wrist flexion, wrist extension, and 2 point pinch) and rest. Muscle synergies account for time-invariant neuromuscular activation of a collection of muscles that contribute to a particular movement. Single muscles can contribute to multiple muscle synergies which when activated over time contribute to movement intention. The extraction of time invariant muscle synergies is performed as disclosed herein, which facilitates providing clinical evaluation for stroke recovery, neuromuscular electrical stimulation pattern (NMES) generation, and domain adaptation. Different methods of dimensionality reduction can be used, including non-negative matrix factorization (NMF), principal component analysis (PCA), independent component analysis (ICA), factor analysis, non-linear methods such as autoencoders or recurrent neural network autoencoders, various combinations thereof, or so forth. By way of muscle synergy analysis, different populations of people can be compared to determine muscle weightings for a particular muscle synergy component for rehabilitation purposes or prognostic evaluation over time. Additionally, muscle synergies can provide an initial pattern for NMES to evoke muscles that work together for a particular movement with a stimulation weighting based on the time-varying activation of the muscle synergy components for the given movement. Still further, a time-invariant representation of muscles working together can provide a latent representation in which new data from a different session or person can be aligned to for domain adaptation in order to reduce calibration time and increase decoder robustness.

Muscle synergy analysis that is tracked over time throughout rehabilitation training provides a biomarker for the corticomuscular coherence for recovery. By tracking muscle synergy changes over time, a link to cortical plasticity can be established that can indicate the viability for motor recovery. More stable muscle synergies may indicate less plasticity and thus alternative methods to increase cortical plasticity can be used to help with rehabilitation. Additionally, early muscle synergy analysis by comparing patients may highlight areas of focus for rehabilitation based on the relative weights of muscles to the muscle synergies. Muscle synergies are similar between arms (or more generally target anatomy) of a patient who suffered a stroke in which one arm is paralytic and the other is functioning normally. By comparing the muscle synergies extracted between both arms, evidence is provided for areas muscles or functional movements to focus on for training.

Functional electrical stimulation (FES) can be used to evoke motor function. However, where to optimally stimulate muscles can vary between people, the detailed configuration of the sleeve 10 and positioning of the electrodes 12, and individual neuro-cognitive impairment. Understanding how the muscles work together to evoke movements can provide a starting calibration position for NMES. Muscle synergies made up of multiple muscles can assist in determining where to stimulate for a given movement. The time-varying activation function can help determine a relative activation of the NMES calibration pattern based on the muscle synergy. Clinical evidence suggests that delivering electrical stimulation such as NMES or functional electrical stimulation (FES) in physiologically relevant patterns (such as based on motor synergies) can provide more useful feedback to the central nervous system during rehabilitation to improve recovery after neurological injury such as stroke. Therefore, this method can assist in developing and delivering FES patterns that are more effective for use during neurorehabilitation such during stroke rehab. Still further, this approach can provide an initial starting point or region to target for an automatic calibration software that can find an optimal stimulation pattern to help evoke movement.

In embodiments disclosed herein, muscle synergies are extracted using the high-density EMG electrode array 12. The muscle synergies provide a biomarker for corticospinal plasticity and motor recovery to help understand and enhance rehabilitation. The use of muscle synergies provides for targeted electrical stimulation activation for different movements specific to the participant. Using approaches such as the network analysis on the similarity matrix to generate a network representing the similarity matrix, unseen data are mapped to known muscle synergies to help increase decoder robustness and reduce calibration time between sessions and people.

This approach has been reduced to practice to produce the data presented in FIGS. 21 and 22A-22B. In one suitable approach, EMG data from a high-density EMG or EMG+NMES electrode array is recorded across various categories of people (able-bodied, stroke, SCI) across multiple functional movements. The EMG data is reduced dimensionally using one or more of the following techniques: linear methods such as NMF, factor analysis, PCA, ICA, compressive sensing or non-linear methods such as autoencoders (variational, recurrent), generative adversarial network, a combination of the techniques or any other dimensionality reduction technique. The reduced data breaks up into time-invariant muscle synergies and corresponding time-varying activation functions. The time-invariant muscle synergy is a combination of multiple muscles. The time-varying activation function activates the muscle synergy over time to reproduce the movement intent. The number of muscle synergies is determined based on how well the original data (i.e. the acquired EMG data) can be constructed from the latent representation (i.e., the reduced-dimensionality data) of muscle activity via the muscle synergies and muscle synergy activation (e.g. able to reproduce original data with greater than 95% variance accounted for (VAF)).

The extracted muscle synergies can be used in various ways. In clinical evaluation, muscle synergies between different arms (or more generally target anatomy) and/or different groups of people can be compared based on weighting to muscles or activation during certain movements to target muscles and/or functional movements for rehabilitation training. The muscle synergy can be correlated with long-term changes to corticospinal reorganization to use as a proxy for motor recovery.

In assistive technology such as NMES, the extracted muscle synergies can be used to: determine which muscle synergies are active for individual movements; initialize starting FES stimulation patterns to the location of muscle synergy pattern; weight the activation of NMES based on the activation of the muscle synergy; and/or use in auto-calibration software to refine evoked movement parameters. For example, FES can be applied to the arm or other target anatomy using the starting FES stimulation pattern.

In NMES used for neurorehabilitation, the extracted muscle synergies can be used to: extract muscle synergies in both arms; stimulate paralytic arm based on functioning arm muscle synergy pattern and activation or based on able bodied muscle synergy pattern and activation; and/or develop NMES patterns based on an individual's muscle synergies to provide physiologically relevant feedback to improve recovery after neurological injury such as stroke.

In domain adaptation, the extracted muscle synergies can be used to; extract muscle synergies from initial calibration data; train the EMG decoder on muscle synergy activation; align new session data to muscle synergies extracted in the calibration session; use the original EMG decoder to test on new aligned data; and combine the aligned data after a session to muscle synergies and the original muscle synergies and train new EMG decoder to increase robustness. This can be repeated after each session.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A method of neurological assessment comprising:
acquiring multichannel electromyography (EMG) data for an anatomical region;
generating a pairwise EMG channel-EMG channel similarity matrix from the acquired multichannel EMG data;
performing network analysis on the similarity matrix to generate a network representing the similarity matrix;
computing one or more metrics of the network; and
determining one or more biomarkers for the anatomical region based on the one or more metrics.

2. The method of claim 1 wherein the acquiring of the multichannel EMG data for the anatomical region comprises acquiring the multichannel EMG data using electrodes disposed in a garment worn on the anatomical region.

3. The method of claim 1 wherein the generating of the similarity matrix includes binarizing the elements of the similarity matrix.

4. The method of claim 1 wherein the network analysis comprises a coherence network analysis.

5. The method of claim 1 wherein the network analysis comprises a correlation network analysis.

6. The method of claim 1 wherein the one or more metrics of the network include one or more network metrics.

7. The method of claim 1 wherein the one or more network metrics include one or more of a density metric measuring a fraction of present connections to possible connections, a global efficiency metric measuring an average inverse shortest path length in the network, a characteristic path length metric measuring an average shortest path length in the network, and/or a core periphery q-stat metric.

8. The method of claim 1 wherein the one or more metrics of the network include one or more nodal metrics.

9. The method of claim 1 wherein the one or more nodal metrics include one or more of a degree metric measuring a number of links connected to a node of the network, a clustering coefficient metric measuring a fraction of neighbors of a node of the network that are neighbors of each other, a local efficiency metric measuring a global efficiency computed on a neighborhood of a node of the network, and/or a betweenness centrality metric measuring a fraction of all shortest paths in the network that contain a node of the network.

10. A method comprising:
acquiring electromyography (EMG) data using an electrode array contacting skin of a target anatomy;
processing the EMG data to produce reduced-dimensionality data; and
determining time-invariant muscle synergies and corresponding time-varying activation functions in the reduced-dimensionality data.

11. The method of claim 10 wherein the processing of the EMG data to produce reduced-dimensionality data comprises processing the EMG data using one or more of: non-negative matrix factorization (NMF); factor analysis; principal component analysis (PCA), independent component analysis (ICA), an autoencoder, a generative adversarial network, or a combination thereof.

12. The method of claim 10 wherein the determining of the time-invariant muscle synergies includes:
determining a number of muscle synergies based on reconstruction of the acquired EMG data from the reduced-dimensionality data via the muscle synergies and muscle synergy activation.

13. The method of claim 10 wherein the reconstruction of the acquired EMG data from the reduced-dimensionality data comprises reproducing the acquired EMG data with greater than 95% variance accounted for (VAF).

14. The method of claim 10 further comprising:
repeating the acquiring, processing, and determining for different anatomical targets and/or different subjects; and
comparing the determined muscle synergies of the different anatomical targets and/or different groups of people to identify target muscles and/or functional movements for rehabilitation training.

15. The method of claim 10 further comprising:
repeating the acquiring, processing, and determining for multiple sessions; and
correlating the determined muscle synergies over the multiple sessions with changes to corticospinal reorganization to assess motor recovery.

16. The method of claim 10 further comprising:
determining a starting stimulation pattern based on the determined muscle synergies; and
performing functional electrical stimulation (FES) or neuromuscular electrical stimulation (NMES) on the target anatomy using the starting stimulation pattern.
